(19) United States
(12) Patent Application Publication
(10) Pub. No.: US 2023/0233802 A1
(43) Pub. Date: Jul. 27, 2023

(54) ACCESS DEVICE HUB

(71) Applicant: ABIOMED, Inc., Danvers, MA (US)

(72) Inventors: Adam Finck, Danvers, MA (US); Emilia Jahangir, Danvers, MA (US); Charles Anthony DeLorenzo, Danvers, MA (US); Glen Fantuzzi, Danvers, MA (US); Zhenghog Tao, Danvers, MA (US); Jiafeng Zhang, Danvers, MA (US); Leo Tse, Danvers, MA (US); Ryan Orizondo, Danvers, MA (US)

(73) Assignee: ABIOMED, Inc., Danvers, MA (US)

(21) Appl. No.: 18/094,067

(22) Filed: Jan. 6, 2023

Related U.S. Application Data
(60) Provisional application No. 63/297,516, filed on Jan. 7, 2022.
(60) Provisional application No. 63/328,184, filed on Apr. 6, 2022.
(60) Provisional application No. 63/344,408, filed on May 20, 2022.

(51) Int. Cl. A61M 25/00; A61M 60/113; A61M 60/38; A61M 60/295; A61M 1/36

(52) U.S. Cl. CPC A61M 25/0097; A61M 60/113; A61M 60/38; A61M 60/295; A61M 1/3666; A61M 1/3659; A61M 2039/0258

(57) ABSTRACT

An access device may be provided that includes a hub configured to improve the flow of blood within a lumen, in order to prevent, e.g., thrombus formation, while also avoiding hemolysis. The hub may have a removably attachable second arm, protrusions/depressions with the lumens of the hub or system, and/or plugs or plug analogs may be used to prevent flow from entering certain regions of the lumens within the hub. These hubs may be used, e.g., as part of an access device, which may have a modular configuration.

### CROSS-REFERENCE TO RELATED APPLICATIONS

The present application claims priority to U.S. Provisional Patent Application Nos. 63/297,516, filed Jan. 7, 2022, 63/328,184, filed Apr. 6, 2022, and 63/344,408, filed May 20, 2022, the entirety of each of which is incorporated by reference herein.

### TECHNICAL FIELD

The present disclosure is drawn to surgical access devices, and specifically to an access device capable of being used to facilitate the introduction of medical devices into a patient and to facilitate the circulation of blood through an extracorporeal device.

### BACKGROUND

Extracorporeal Membrane Oxygenation (ECMO) involves the use of mechanical circulatory device for patients experiencing cardiogenic shock, or other forms of hemodynamic deterioration. Ventricular assist device (VADs) and catheter based VADs (such as intravascular blood pumps) may be used to unload the heart (e.g., the left ventricle).

Access devices, which generally include a cannula attached to a hub, are commonly used in surgical procedures to facilitate the introduction of a medical instruments into the body's natural biological blood vessels, cavities, etc. These access devices include, for example, devices that facilitate the introduction of guide wires, balloon catheter, or intravascular blood pumps (such as a catheter-based heart pump) into the vasculature of the human body. These access devices also can be used to facilitate the extracorporeal circulation of blood, such as when utilizing an extracorporeal membrane oxygenation (ECMO) device (including, e.g., veno-arterial ECMO (VA-ECMO) or veno-venous ECMO (VV-ECMO) devices).

### BRIEF SUMMARY

According to a first aspect of the present disclosure, a hub can be provided that improves the flow of a fluid within the hub, minimizing the risk of thrombus formation and hemolysis.

In some embodiments, the hub may include a first arm with a first lumen extending from a proximal end to a distal end, the distal end being configured to be operably coupled to a cannula. In some embodiments, the first lumen may be non-linear, such that a central axis of the first lumen at the distal end forms an angle with the central axis of the first lumen at the proximal end. In some embodiments, the first arm may be configured to be operably coupled to a second arm, the second arm may include a second lumen extending therethrough. In some embodiments, the second arm may be coupled to the hub, and may be removably coupled to the hub. In some embodiments, the hub may include a protrusion, a depression, or both within the first lumen, a protrusion, a depression, or both with the second lumen, or a combination thereof.

In some embodiments, a plug may be used, where hub may be configured to removably receive the plug through a proximal end of the first lumen, such that at least a portion of the first lumen is blocked (e.g., where a fluid cannot enter the blocked portion). The plug may be configured to be removably inserted into a proximal end of the first lumen. In some embodiments, the plug may include collagen. In some embodiments, a plug analog may be used. For example, in some embodiments, one or more dilators may be used, where the dilators are configured to extend from the proximal end of the first lumen less than an entire length of the first lumen. In some embodiments, the dilator(s) may be configured to fill at least a portion of space in the first lumen at or near the proximal end of the first lumen. In some embodiments, a plug may be configured to be removably inserted into a proximal end of the second lumen.

In some embodiments, the plug may include a slit through which a medical device may be inserted into the first lumen. In various embodiments, the medical device may be, e.g., a guide wire, a balloon catheter, or a catheter-based heart pump. In some embodiments, the one or more dilators may be configured to extend through the slit.

In some embodiments, a hemostatic valve may be disposed in the first arm. In some embodiments, the hemostatic valve may be positioned adjacent to the plug.

In some embodiments, the hub may include a third lumen operably connected to the first lumen, the second lumen, or both. The third lumen may be configured to, e.g., connect to an external accessory, such as a distal leg perfusion cannula, a pressure bag, or an infusion pump. In some embodiments, the third lumen is configured to allow a fluid to enter or exit the cannula through the hub. In some embodiments, the third lumen may be connected to a valve.

In some embodiments, the first arm may be perpendicular to the second arm. In some embodiments, the first arm may extend tangentially to the second arm. In some embodiments, the first lumen of the first arm may extend tangentially to the second lumen of the second arm. In some embodiments, a longitudinal axis of the first arm may be laterally offset from a longitudinal axis of the second arm. In some embodiments, the central axis of the distal portion of the first arm, and the central axis of the second arm, may form an angle at the proximal end of the hub, where the angle is 15-30 degrees.

In some embodiments, a cap may be coupled to a proximal end of the first arm. In some embodiments, an O-ring and a silicone valve may be positioned between a portion of the cap and a portion of the first arm. In some embodiments, a second O-ring may be disposed between the silicone valve and the cap. In some embodiments, the cap may be a Touhy Borst valve.

According to a second aspect of the present disclosure, an access device that includes the hub may be provided. The access device may include a cannula and any embodiment of the hub as disclosed herein, the hub being configured to be coupled to a proximal end of the cannula. In some embodiments, the cannula may be coupled to the hub via a threaded connection. In some embodiments, the cannula may have a wall thickness of between 0.2 mm and 0.4 mm. In some embodiments, the cannula may be reinforced with coiled wire, braided wire, or a precision-cut hypotube. In some embodiments, the cannula may include a low-friction polymer coating on an inner surface of the joint lumen, such as Polytetrafluoroethylene (PTFE). In some embodiments, the cannula may utilize a thermoplastic polyurethane, a nylon, or a polyamide block polymer. In some embodiments, the cannula may utilize a radiopaque material. In some embodiments, the cannula may include a straight cannula. In some embodiments, the cannula may be configured to receive a dilator assembly.

In some embodiments, the access device may include a second arm configured to be removably coupled to the hub. The second arm may include a second arm with a second lumen extending therethrough, the second lumen configured to be operably coupled to the first lumen through an opening in the hub.

In some embodiments, the access device may include a tubular extension. The tubular extension may be configured to be removably coupled to a proximal end of the cannula, and for the hub to be removably coupled to a proximal end of the tubular extension. In some embodiments, the tubular extension may also be configured to be removably coupled to a proximal end of a second arm.

In some embodiments, the access device may include a clamp configured to allow a user to clamp off the second arm. In some embodiments, the access device may include a fixation feature. In some embodiments, the fixation feature may be a butterfly pad or a suture ring. In some embodiments, the fixation feature may be axially stationary with respect to the cannula. In some embodiments, the fixation feature may be movably positioned along the cannula.

In some embodiments, the access device may include one or more caps coupled to the proximal end of the hub. The one or more caps may include a Touhy Borst valve. In some embodiments, the access device may include a silicone valve and at least O-ring “sandwiched” between the hub and one of the caps, or between two caps, where the compression causes the O-ring(s) to deform and aid in the barrier function of the silicone valve. In some embodiments, a deformed O-ring acts as a first barrier to resist pressure of blood within the hub. In some embodiments, a deformed O-ring is position proximal to the silicone valve and, by deforming, supports the silicone valve.

In some embodiments, the first arm may extend tangentially to the second arm. In some embodiments, a lumen of the first arm may extend tangentially to that of a second lumen of the second arm. In some embodiments, the first arm may extend perpendicularly to the second arm.

According to a third aspect of the present disclosure, a method of using the hubs and access devices disclosed herein may be provided. The method may include providing an access device according to any of the embodiments disclosed herein and inserting the cannula of the access device into a patient. Then, the method may include inserting a medical device through a hub and into the patient, and/or oxygenating blood with an extracorporeal membrane oxygenation (ECMO) device operably coupled to the cannula through a second arm, an alternate connector subsystem, or both. In some embodiments, the medical device is inserted through a hemostatic valve, a first lumen, and a joint lumen. In some embodiments, the medical device is inserted through a hemostatic valve, a first lumen, the alternate connector subsystem, and a joint lumen. In some embodiments, the medical device may be an intravascular blood pump.

According to a fourth aspect of the present disclosure, a kit may be provided. The kit may include an access device according to any of the embodiments disclosed herein, an extracorporeal membrane oxygenation (ECMO) device configured to be operably coupled to a cannula of the access device, and a medical device configured to be inserted through a hemostatic valve, a first lumen, and a joint lumen of the access device. In some embodiments, the medical device may be an intravascular pump.

### BRIEF DESCRIPTION OF DRAWINGS

FIG. 1 is a cutaway view of an embodiment of an access device.

FIGS. 2, 3, 4A-4B, 5, and 6 are cutaway views of embodiments of hubs.

FIG. 7 is a cutaway view of an embodiment of an access device configured as a modular system.

FIGS. 8A-8B are cutaway views of embodiments of hubs.

FIGS. 9A-9D are cutaway views of embodiments of different configurations of an access device configured as a modular system.

FIGS. 10A and 10B are cutaway views of embodiments of different connection options for a hub.

FIG. 11 is a flowchart of an embodiment of a method.

FIG. 12 is a schematic of an embodiment of an access device inserted into a patient.

FIG. 13 is a perspective view of an embodiment of a hub.

FIG. 14 is a side view of the hub of FIG. 13.

FIG. 15 is a top view of the hub of FIG. 13.

FIG. 16 is a cross-sectional view of the hub of FIG. 13.

FIG. 17 is a top view of a hub according to another embodiment.

FIG. 18 is an illustration of an embodiment of a system.

FIG. 19A is a side view of an embodiment of a hub.

FIG. 19B is an exploded side view of an embodiment of a hub.

FIG. 19C-19E are cross-sectional side views of an embodiment of valves in a hub, showing an uncompressed view with a single O-ring (19C), a compressed view with a single O-ring (19D), and a compressed view with two O-rings (19E).

### DETAILED DESCRIPTION

Cardiogenic shock is the leading cause of death for patients with acute myocardial infarction (AMI) who reach the hospital alive. Cardiogenic shock is caused by a heart malfunction or problem, which leads to an inability of the heart to eject enough blood for the body. In some instances, ventricular assist devices (VADS) and catheter-based VADS (such as intravascular blood pumps) may be used to mechanically unload the heart (e.g., the left ventricle).

Extracorporeal Membrane Oxygenation (ECMO) allows for gas exchange of the blood when the lungs do not work properly and may involve the use of a mechanical circulatory device for patients experiencing oxygenation issues. In some instances, ECMO may be used for patients experiencing oxygenation issues due to cardiogenic shock, or from other forms of hemodynamic deterioration. In some instances, use of such devices may result in an increase in left ventricular afterload.

As described herein, in some instances, patients may need both ECMO support and a VAD. In some instances, such support may take place at the same time, although in some instances, a patient may require ECMO support prior to and/or after VAD support. Traditionally, this requires multiple insertion points, which may add additional time, complexity, and/or risks to a surgical procedure. As such, the inventors have recognized the benefit of an access device capable of being used to facilitate the introduction of multiple medical devices.

The inventors have also recognized the benefits of improving flow through the access device so as to minimize and/or prevent stagnation of blood within the access device. In some embodiments, this may minimize the risk of thrombus formation within the access device. The inventors have also recognized the benefit of improving flow through the access device so as to minimize the risk of hemolysis due to turbulent flow.

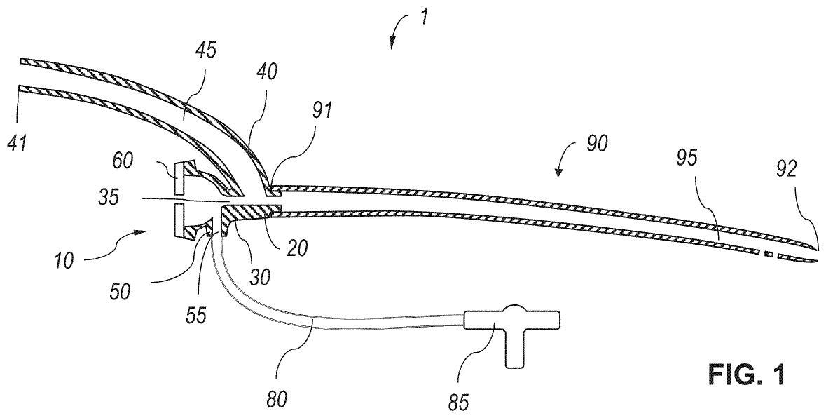

Referring to FIG. 1, an access device 1 according to embodiments of the present disclosure can be seen. As shown in this view, in some embodiments, the access device 1 generally includes first arm 30, which may allow one or more medical devices to be inserted through the device into a patient and a second arm 40 which may allow an external medical device (e.g., an ECMO device) to be connected to the patient for support. As will be appreciated, in some embodiments, the second arm also may allow for insertion of one or more medical devices. As will be further appreciated, various medical devices may be utilized. For example, in some embodiments, catheter-based medical devices may be inserted into the patient.

As described herein, the access device may allow for simultaneous ECMO support and insertion of medical device and tandem ECMO support and insertion of a medical device (e.g., insertion before and/or after completion of ECMO support). The device also may allow for a medical device (e.g., a VAD) to remain installed in a patient and through the access device while ECMO support is discontinued.

In some embodiments, the access device 1 may include a hub 10 having a hub body 20 which defines the first and second arms 30, 40. In some embodiments, the hub 10 is coupled to a shared cannula 90 at a proximal end 91 of the cannula, the cannula defining a joint lumen 95 (sometimes referred to as a shared lumen) therethrough. As will be appreciated, the cannula may be permanently attached to the access device or may be attachable to the access device (e.g., by a clinician). In embodiments in which the cannula is attachable to the access device, the cannula may be configured to be fixedly attached to the access device for insertion into a patient.

For purposes herein, the joint lumen may include a single lumen extending along the length of the shared cannula that may be used to both pass one or more medical devices and to pass blood therethrough (e.g., from an ECMO circuit). In other embodiments, the joint lumen may include more than one lumen extending along the length of the shared cannula. For example, in some embodiments, the cannula may include two parallel lumens extending along the length of the shared cannula. In such embodiments, the medical devices may extend through a first lumen and the ECMO circuit may be operably connected to the second circuit. In another embodiment, the shared cannula may include a first portion with a single lumen and a second portion with more than one lumen (e.g., two parallel lumens). In such embodiments, the single lumen may communicate with each of the two lumens.

Although shown and described as being attached to a shared cannula, it will be appreciated that the access device may be attached to the patient via other suitable manners. For example, in some embodiments, the access device may be connected to a graft, which is thereafter attached to the patient.

As shown in FIG. 1, the first arm 30 may define a first lumen 35 therethrough, and the second arm 40 may define a second lumen 45 therethrough. In some embodiments, the second lumen 45 may intersect the first lumen 35. In some embodiments, the first arm may include a hemostatic valve 60 arranged to minimize and/or prevent blood leakage via the first arm. In some embodiments, the hemostatic valve may be located at the proximal end of the first arm. In some embodiments, the first arm may include more than one hemostatic valve, such as two hemostatic valves (e.g., first and second hemostatic valves). In some embodiments, a medical device may be passed through the hemostatic valve 60, through the first lumen, through the joint lumen 95, and out a distal end 92 of the cannula (and into the patient). In some embodiments, the medical device to be inserted may be a guide wire, a balloon catheter, or a catheter-based heart pump. As will be appreciated, other catheter-based medical devices also may be insertable via the first arm. In some embodiments, the medical device to be inserted may be an intravascular heart pump. In some embodiments, a portion of each of a plurality of medical devices may be present within the first lumen and joint lumen of the access device at the same time.

In some embodiments, an external medical device, such as an ECMO device (not shown) may be operably coupled to a proximal end 41 of the second arm, and blood may flow through the second lumen 45 and the joint lumen 95, and out of the distal end 92 of the cannula into the patient. In some embodiments, the access device may include a clamp configured to allow a user to clamp off the second arm, such as to control blood flow into and out of the access device. In some embodiments, the clamp may be integral to the second arm, although the clamp also may be removably attached to the second arm. In some embodiments, the clamp may include a Roberts clamp, although other suitable clamps may be used in other embodiments. It will be appreciated that the second arm may include other arrangements for controlling blood flow through the second arm. For example, in some embodiments, the second arm may include built-in valving (e.g., a stop cock) or clamping to control flow.

In some embodiments, the hub 10 also may include a third arm 50 that defines a third lumen 55 therethrough. The third lumen 55 also may intersect the first lumen 35 in some embodiments. As will be appreciated, the third lumen may be connected to the first lumen, the second lumen, or both. In some embodiments, the third lumen 55 may be configured to connect to an external accessory, such as a distal leg perfusion cannula, a pressure bag, or an infusion pump. In some embodiments, the third lumen 55 may be configured to allow a fluid to enter or exit the cannula 90 through the hub 10. In some embodiments, the third lumen 55 may be coupled to tubing 80. In some embodiments, the third lumen 55 may be connected, directly or indirectly, to a valve 85. In some embodiments, the valve 85 may be between the hub 10 and an external accessory (not shown). In some embodiments, the valve may be a three-way stopcock.

According to some embodiments, the hub may be configured to reduce thrombus formation and turbulent flow risks to an acceptable level consistent with ISO standards. In some embodiments, as described herein, the hub may be optimized to minimize regions in the hub where blood can stagnate. In some embodiments, this may include optimizing the flow path of blood and/or minimize identified stagnation regions. In some embodiments, this may include at least partially plugging one or more regions in which blood may stagnate. In other embodiments, as described herein, this may include configuring the hub such that the blood flow may wash out one or more portions of the hub (e.g., a portion of the lumen or a portion of a side of the hemostatic valve) as the blood travels through the hub and/or one or more lumens and into the patient.

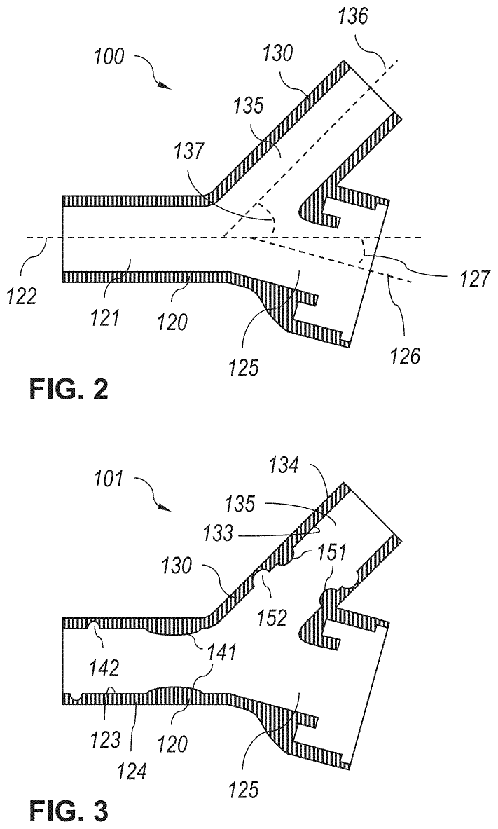

Referring to FIG. 2, in some embodiments of the hub 100, the first lumen may include a distal portion 121 and a proximal portion 125. In some embodiments, a central axis 126 of the proximal portion 125 may form a first arm angle 127 with the central axis 122 of the distal portion 121, where the first arm angle 127 may be 15 degrees-145 degrees. In some embodiments, the angle may be 15 degrees-30 degrees. In some embodiments, the central axis 136 of the second lumen 135 may form a second arm angle 137 with the central axis 122 of the distal portion 121, where the second arm angle 137 may be 15 degrees-145 degrees. In some embodiments, the second arm angle may be 15 degrees to 30 degrees. In some embodiments, the second arm angle may be 30 degrees. In some embodiments, the second arm angle 137 may be greater than the first arm angle 127. In some embodiments, the second arm angle 137 may be less than the first arm angle 127.

Referring to FIG. 3, in some embodiments, the hub 101 may include one or more protrusions 141 extending inwardly from an internal surface 123 of the hub and into the flow path (e.g., into the flow path of the first and/or second lumens). In other embodiments, the internal surface 123 of the hub may include one or more depressions 142 in the sidewall 124. In some embodiments, the hub may include one or more protrusions and one or more depressions on an internal surface. That is, the first and/or second arm may include depressions and protrusions on its respective internal surface. As will be appreciated, the first and second arms need not have the same number of protrusions and/or depressions on the internal surface.

As will be further appreciated, the shape and/or size of the protrusions and depressions may be the same or may vary from protrusion to protrusion or from depression to depression. For example, as shown in FIG. 3, the first arm includes two larger protrusions while the second arm includes two smaller protrusions. The second arm also has two larger depressions as compared to the size of the depressions of the first arm. In some embodiments, the protrusions and/or depressions may be symmetrically positioned along the length of the first and/or second arm. In some embodiments, some or all of the protrusions and/or depressions may be asymmetrically positioned along the length of the first and/or second arm. In some embodiments, the protrusions and/or depressions may be configured to control the flow of a fluid (e.g., blood) through the hub. Accordingly, the shape, size, number, and position of the depressions/protrusions may be selected to obtain a desired flow path of blood from the second arm (e.g., and the ECMO circuit), into the distal region of the first arm, and into the joint cannula.

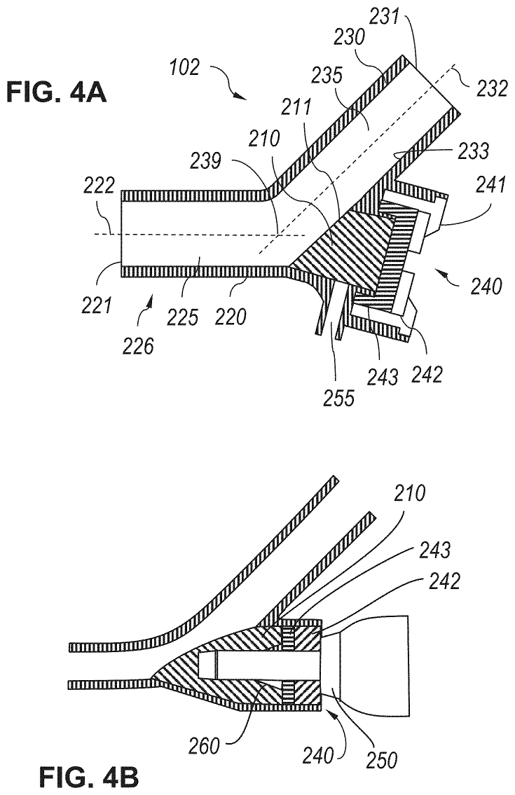

In some embodiments, blood may stagnate in portions of the lumen of the first arm. Accordingly, as shown in FIGS. 4A and 4B, in some embodiments, the hub 102 may include a plug 210 configured to plug at least a portion of the first lumen 225 to minimize and/or prevent such stagnation regions. In some embodiments, the plug 210 may be configured to prevent fluid from flowing back towards a proximal end portion 240 of the first arm when fluid flows from a proximal end 231 of the second lumen 235 and into the first arm 220 before flowing out of a distal end 221 of the first lumen and into the patient. In some embodiments, the proximal end portion 240 of the first arm may include one or more hemostatic valves, such as hemostatic valve 243. In some embodiments, the proximal end portion 240 also may include an end cap 241 and a foam member 242. As shown in this view, the hemostatic valve may be sandwiched between the plug and the foam member. In some embodiments, the end cap, the foam member, and the hemostatic valve may be fixedly attached to one another and attachable to the first arm. As will be appreciated, such components also may be attached to each other in other suitable manners.

In some embodiments, the plug 210 may be configured to fill some or all of the space extending from the proximal end portion 240 towards a point at which the second lumen fully enters the first lumen. In some embodiments, the plug 210 may be configured to fill some or all of the space between the proximal end portion 240 and a point 239 at which the central axis 232 of the second lumen 235 intersects the central axis 222 of a distal portion 226 of the first lumen 225. In some embodiments, the plug 210 may have a surface 211 that is tangential to an internal surface of the second arm 230. In such embodiments, the plug may cooperate with the first and second arms to form a smooth flow path along which blood may travel.

In some embodiments, the plug 210 also may block the third lumen 255.

In some embodiments, as shown in FIG. 4B, the plug may include a slit 260 through which a medical device may pass (e.g., from the first arm and into the joint lumen). In this regard, in such embodiments, the plug 210 may also serve as a hemostatic valve. In some embodiments, this may provide redundancy to the hydrostatic valve 243 in the proximal end portion to minimize and/or prevent blood leakage out of the first arm.

In some embodiments, the plug may be removably attached to the hub. In some embodiments, the plug may be pressure fit into the first arm. In some embodiments, the plug may be screwed into place and/or locked into place.

In some embodiments, the plug may include a collagen material. In some embodiments, the collagen may be irreversibly hydrolyzed. In some embodiments, the plug may include an elastomeric material.

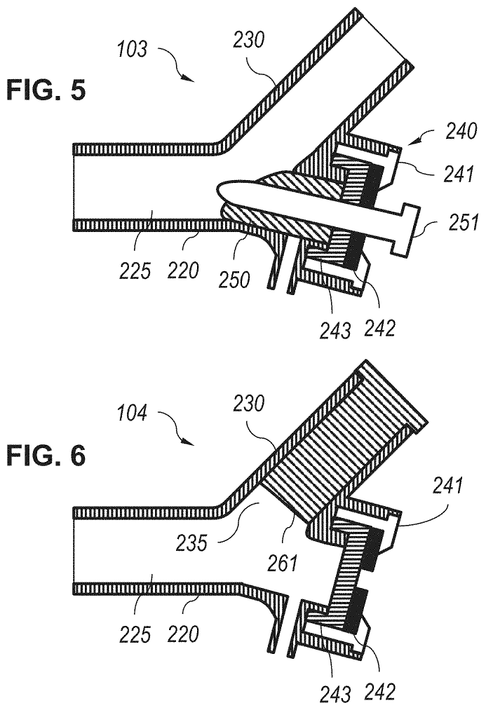

Referring to FIGS. 4B and 5, in some embodiments, instead of or in addition to the plug, one or more dilators 250, 251 may be used to prevent fluid from flowing back towards the proximal end portion 240 of the first arm when fluid flows from a proximal end 231 of the second lumen 235 and into the first arm 220 before flowing out of a distal end 221 of the first lumen. In some embodiments, like that shown in FIG. 4B, dilator 250 may pass through the slit 260 in the plug 210 (and the hemostatic valve 243) where a medical device may otherwise be passed. In these embodiments the dilator may eliminate some or all of the space within the first lumen 225 in which blood may stagnate. In some embodiments, a single dilator may be used. In other embodiments (see, e.g., FIG. 5), a first dilator 250 may be inserted through the hemostatic valve 243 first, and then a second dilator 251 may be inserted into the first dilator. In some embodiments, the shape and size of the cap and/or the proximal end portion may correspond to the shape and size of a portion of the dilator. In some embodiments, the dilator may include a key that is received in a recess at the proximal portion to determine proper insertion of the dilator into the hub. In some embodiments, the dilator is arranged to be locked onto the first arm of the hub so as to maintain the seal via the dilator.

Referring to FIG. 6, in some embodiments, a second plug 261 may be provided that allows for some or all of the second lumen 235 of the second arm to be blocked if the second arm is not operably coupled to an external medical device, such as to an ECMO circuit. In such embodiments, the shape and/or size of the second plug may correspond to that of the second arm. Like the first plugs for the first arm, the second plug 261 may be configured to be locked into place. In some embodiments, the second plug 261 may be screwed into place.

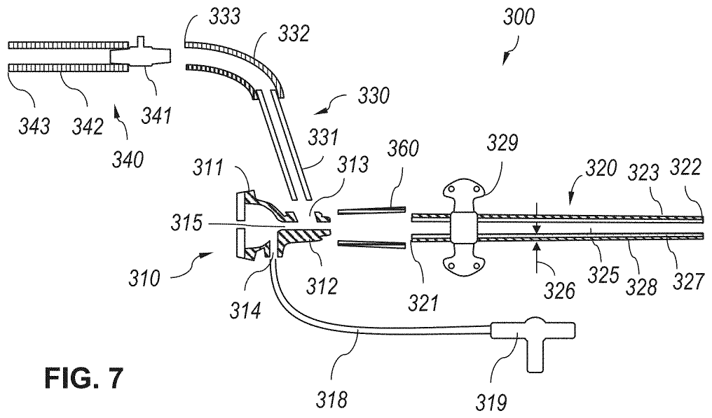

Referring to FIGS. 7, 8A-8B, and 9A-9D, the access device may be configured as a modular system such that the clinician may configure the access device according to the type and/or order of the support needed by the patient. In this regard, the portions of the access device not in use may be removed, which could also remove possible locations in which blood could stagnate. In some embodiments, a kit may be provided that may include all of these modular components.

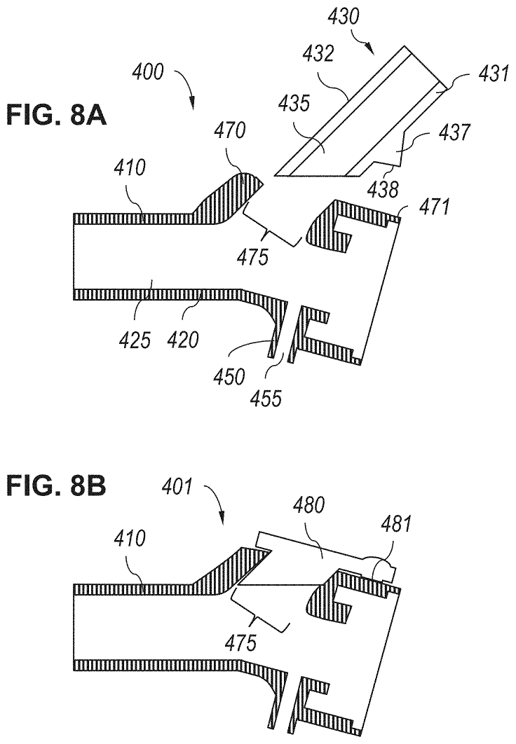

Referring briefly to FIG. 8A, the walls 410 of the hub 400 in the hub subsystem may define a first arm 420 with a first lumen 425 therethrough. In some embodiments, the walls 410 may define an opening 475 at which the second arm may be operably connected to the hub. For example, in some embodiments, the second arm may include a tubular body 431 with a second lumen 435 therethrough that is attached to the hub. In some embodiments, at least a portion of the outer surface 432 of the tubular body 431 may be configured to be inserted into the opening 475. In some embodiments, the outer surface 432 of the tubular body 431 and at least a portion 470 of the walls 410 may form a pressure fit seal. In some embodiments, a portion 470 of the walls 410 contains threads, ridges, or other components that allow the tubular body 431 to be secured into place. In some embodiments, the tubular body may contain one or more keys or protrusions 437 on an outer surface 432. In some embodiments, a distal surface 438 of a protrusion 437 is configured to interact with an outer surface 471 of the hub 400. In some embodiments, the protrusion is configured to allow the second arm to be positioned properly within the opening 475 and/or first lumen 425. In some embodiments, the protrusion also may form a component in a locking mechanism for the second arm.

In some embodiments, the hub also may include a third arm (see, e.g., FIG. 8A, third arm 450) with a third lumen (see, e.g., FIG. 8A, third lumen 455) therethrough. In some embodiments, the third lumen operably connects to the first lumen 425. As shown in FIG. 7, the hub 310 may define a third lumen 314 that is coupled to tubing 318. In some embodiments, the third lumen 314 may be connected, directly or indirectly, to a valve 319. In some embodiments, a valve 319 may be between the hub 310 and an external accessory. In some embodiments, the valve may be a three-way stopcock.

As can be seen in FIG. 8B, in some embodiments, when the second arm is removed, a plug 480 may be provided to removably block the opening 475. In some embodiments, the plug 480 may be fully removable from the hub. In some embodiments, the plug may be coupled to the hub at an attachment point 481, while still allowing the plug to be inserted and removed from the opening 475.

Referring back to FIG. 7, the cannula may include a lumen therethrough, extending from a proximal end 321 to a distal end 322. In some embodiments, a fixation feature 329, such as a butterfly pad or a suture ring, may be attached to the cannula for securement. In some embodiments, the fixation feature may be configured to be axially stationary with respect to the cannula. In some embodiments, the fixation feature may be rotatable about the cannula. In some embodiments, the fixation feature may be movably positioned along the cannula 323.

In some embodiments, the cannula may have a wall thickness 326 of between 0.2 mm and 0.4 mm. In some embodiments, the wall thickness may be substantially constant. In some embodiments, the wall thickness in one portion of the cannula may be thicker than the wall thickness in a different portion of the cannula (excepting any rounded or thinned ends of the cannula).

In some embodiments, the cannula may include one or more layers 327, 328. In some embodiments, the cannula may include an inner layer 327 and an outer layer 328 (sometimes referred to as an outer jacket). In some embodiments, some or all of the cannula may be reinforced with coiled wire, braided wire, or a precision-cut hypotube. In some embodiments, the outer jacket may include coiled wire, braided wire, or a precision-cut hypotube. In some embodiments, the cannula may include a low-friction polymer coating (such as Polytetrafluoroethylene (PTFE)) on an inner surface of the joint lumen. In some embodiments, the inner layer may include a low-friction polymer coating (such as Polytetrafluoroethylene (PTFE)). In some embodiments, one or more of the layers forming the cannula may include a thermoplastic polyurethane, a nylon, or a polyamide block polymer.

In some embodiments, the cannula may include a radiopaque material. In some embodiments, the radiopaque material is a metallic element. In some embodiments, the radiopaque material is tungsten, silver, tantalum, or tin. In some embodiments, the radiopaque material is a tungsten powder. In some embodiments, the radiopaque material may be combined with a polymer (such as a polyurethane). In some embodiments, the radiopaque material is arranged in bands offset axially from each other along some or all of the length of the cannula. [0051] In some embodiments, as shown in FIG. 7, the modular system may include a tubular extension 360 for attaching the cannula to different hubs. As will be appreciated, in some embodiments, the hubs may be attachable directly to the cannula.

In some embodiments, the cannula may be configured to receive a dilator assembly.

In some embodiments, the second arm second arm 331 may be coupled to flexible tubing 332, to a connector 341, and to an external medical device, such as an ECMO circuit (e.g., via flexible tubing 342). For example, the proximal end 343 of the flexible tubing 342 may be configured to be removably coupled from the medical device (such as an ECMO, heat exchanger, etc.) when used in one configuration, and the hub 311 when used in a different configuration. A clamp 362 may be used in some embodiments to control the flow of a fluid through flexible tubing 332.

In some embodiments, the fitting or connector 341 is configured to allow the alternate connector subsystem 340 to be removably coupled to a proximal end 333 of the second arm subsystem 330 in when used in one configuration and removably coupled to a proximal end 321 of the cannula subsystem 320 when used in a different configuration. In some embodiments, the fitting or connector 341 is configured to allow the alternate connector subsystem 340 to be removably coupled or removably coupled to a proximal end 361 of the tubular extension 360.

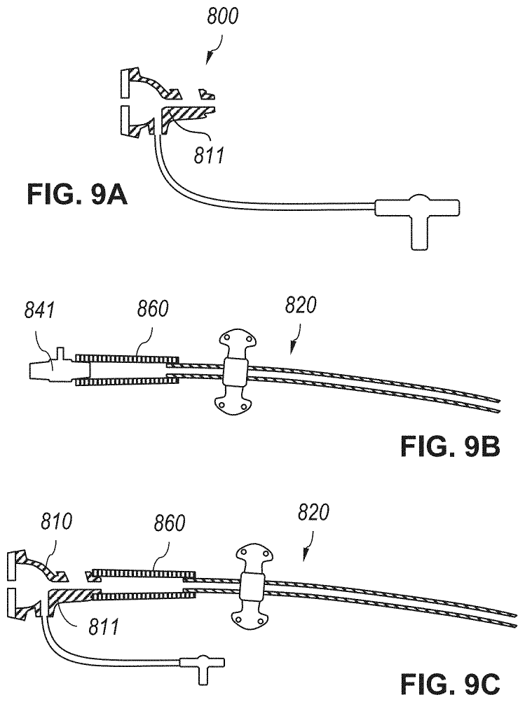

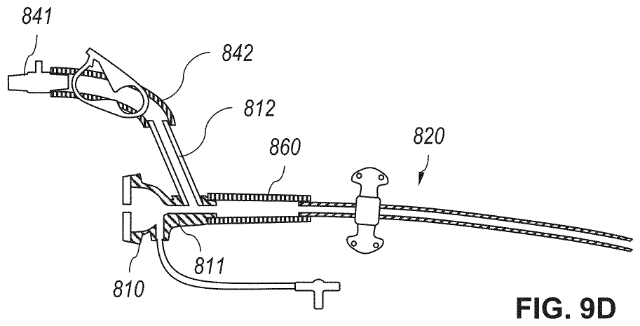

The modular access device may be configured such that one or both arms are removably attachable to the hub. For example, as shown in FIG. 9A, in some embodiments of a modular access system 800, the second arm (e.g., connected to the ECMO circuit) may be removable from the hub 810, such as after ECMO support has been completed. In such embodiments, the first arm 811 may remain attached to the hub while the patient is under VAD support. In a similar fashion, a clinician may begin using the hub with only the first arm attached when VAD support is needed, and then attach the second arm if/when ECMO support is then needed. In other embodiments, the clinician may attach just the second arm to the hub if only ECMO support is needed first, and thereafter attach the first arm to the hub if/when VAD support is needed. As will be appreciated in view of the above, the clinician may still decide to leave both the first and second arms attached to the hub, irrespective of which type of support is needed by the patient.

FIGS. 9B-9D illustrate embodiments in which a tubular extension 860 can be used to attach different configurations of the modular access device for patient support. For example, as shown in these views, the tubular extension 860 is attachable to the cannula 820, which can be inserted into the patient at the single insertion site (not shown). In embodiments in which only ECMO support is needed (or is needed first) the clinician may attach only a connector 841 to the tubular extension for ECMO support (see FIG. 9B). Once ECMO support has been completed, the connector can be removed, the clinician may attach the hub with only a single arm (e.g., the first arm 811) if/when VAD support is needed (see FIG. 9C). As will be appreciated, the clinician need not first attach the connector 841 (e.g., an ECMO connector) to the tubular extension. Instead, if only VAD support is needed, the clinician can attach just the hub 810 with the first arm 811 to the tubular extension 860 (see FIG. 9C). Finally, as shown in FIG. 9D, the access device with both the first arm 811 and second arm 812 may be attachable to the tubular extension 860 if simultaneous or tandem support is to be provided to a patient. As shown in this view, the connector 841 may be attached or operably coupled to the second arm 812 in some embodiments (e.g., directly or indirectly, such as via flexible tubing 842).

In some embodiments, some components in the system, such as the hub, the second arm, the connectors, and any plugs or dilators, may be configured to be removable, and replaced. In some embodiments, one or more of the components may be removed, cleaned, and reassembled into place.

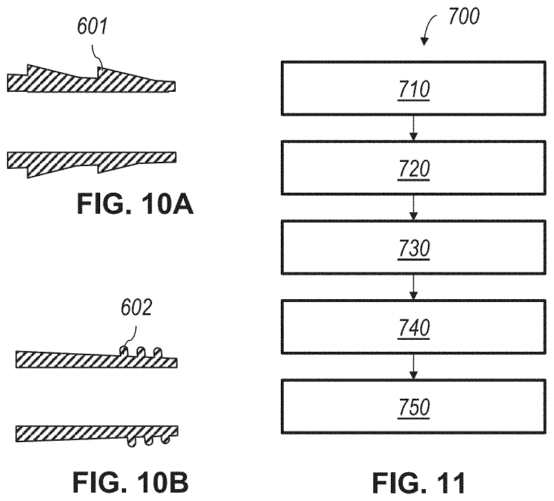

In some embodiments, a hub may be configured to have a smooth connection for the cannula or other component. As seen in FIG. 10A, the parts of the hub that connect to the cannula or other components may include a barbed connection 601. As seen in FIG. 10B, the parts of the hub that connect to the cannula or other components may include a threaded connection 602.

In some embodiments, the hub may be configured to allow a user to visualize flow and thrombus formation within the hub. For example, in some embodiments, the hub may be formed or include a portion which is made from a transparent material. In some embodiments, one or more portions of the hub may include a transparent window allowing a user to see into one or more portions of the first lumen. In some embodiments, one or more portions of the hub may include a transparent window allowing a user to see into one or more portions of the second lumen.

In some embodiments, the access device may include a rigid and a flexible material. In some embodiments, the rigid material (e.g., HIPS, ABS, nylon, etc.) may be used for injection molded elements, while the flexible material (e.g., a thermoplastic polyurethane) may be used for overmolded or insert-molded elements. In some embodiments, the flexible material may be used to couple the cannula to the hub.

According to another embodiment of the present disclosure, a method for using the above-described access devices is provided. Referring to FIG. 11, embodiments of the method 700 may first include providing 710 any embodiment of an access device or system as disclosed herein. This access device or system may be surgically attached to a patient, where at least a portion of the cannula is inserted 720 into the patient through a single insertion site.

The method may then include an insertion step 730, where a medical device (such as an intravascular blood pump, etc.) may be inserted into the first arm of the access device and then into a patient through the cannula. In some embodiment, insertion step 730 may include inserting a medical device through the hemostatic valve, the first lumen, and the joint lumen.

The method also may include coupling 740 an external device, such as the ECMO device, to the second arm of the access device, after which the method may include oxygenating 750 blood with the ECMO device, where the blood flows through the joint lumen and the second lumen of the access device. As will be understood, the insertion step 730 and the coupling step 740 may be completed in any order. In some embodiments, the coupling 740 and oxygenating 750 steps may be completed before the insertion step 730 is completed.

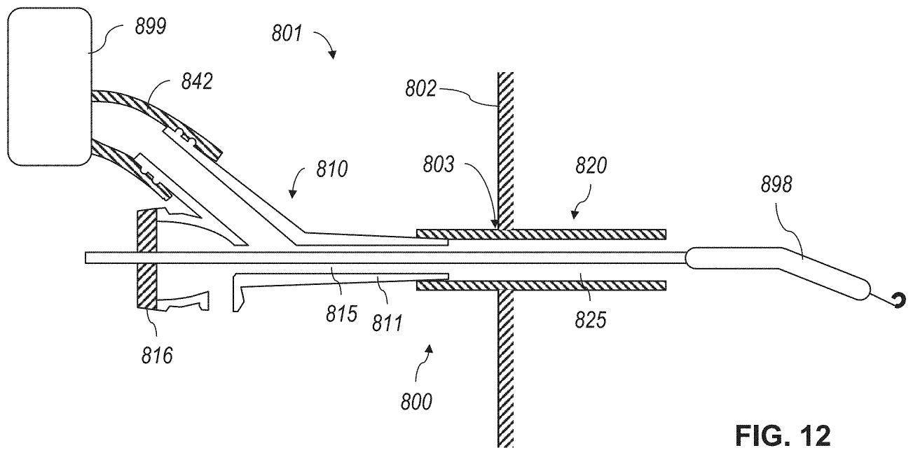

The disclosed method can be seen visually in FIG. 12, where an embodiment of a system 801 may include the modular access system 800 can be seen inserted into a patient. There, at least a portion of the cannula 820, which may be coupled to the hub 810, has been passed through the surface of a patient's skin 802 at an insertion site 803, and into the patient.

A medical device 898 (here, an intravascular blood pump) has been inserted into the first arm 811 and then into a patient through the cannula 820. Specifically, the medical device 898 has been inserted through the hemostatic valve 816, the first lumen 815, and the joint lumen 825.

According to another embodiment of the present disclosure, a kit may be provided. The kit may include any embodiment of an access device according to the first aspect of the present disclosure, an external medical device, such as an extracorporeal membrane oxygenation (ECMO) device, configured to be coupled to the second arm of the single access device, and at least one medical device configured to be inserted through the first hemostatic valve, the first lumen, and the joint lumen of the access device. In some embodiments, the medical device may be an intravascular pump. The kit also may include a cannula attached to the access device. In some embodiments, the kit also may include a needle to enable the physician to gain access to the artery or vein. In some embodiments, the kit also may include a guidewire to enable placement of the cannula into the vasculature. The kit also may include one or more dilators at subsequent sizes to sequentially expand the vascular prior to insertion of the described device.

The basic components of such a kit can be seen in FIG. 12, where there is a first device (e.g., modular access system 800), an external medical device 899 (e.g., an ECMO device), and at least one medical device (e.g., medical device 898) configured to be inserted through a part of the access device.

In some embodiments, the kit may also include additional medical devices, such as one or more dilator assemblies, and/or one or more needles.

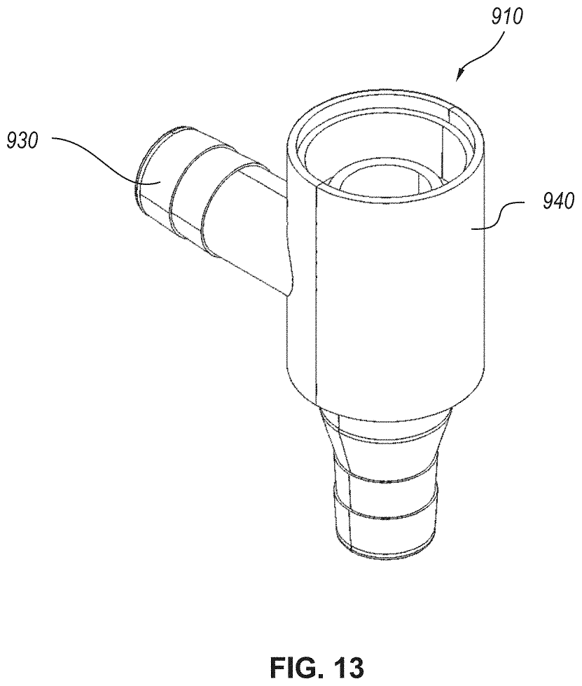

FIGS. 13-15 illustrate a hub 910 according to another embodiment of the present disclosure. As shown in these views, the hub may include a first arm 930 which may allow for ECMO support for a patient, and a second arm 940 which may allow a medical device to be inserted through the hub and into the patient. As with the above, in some embodiments, the second arm may include one or more hemostatic valves.

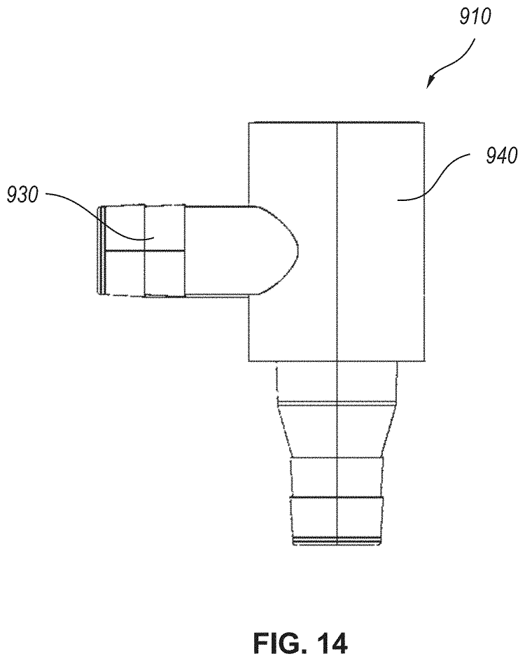

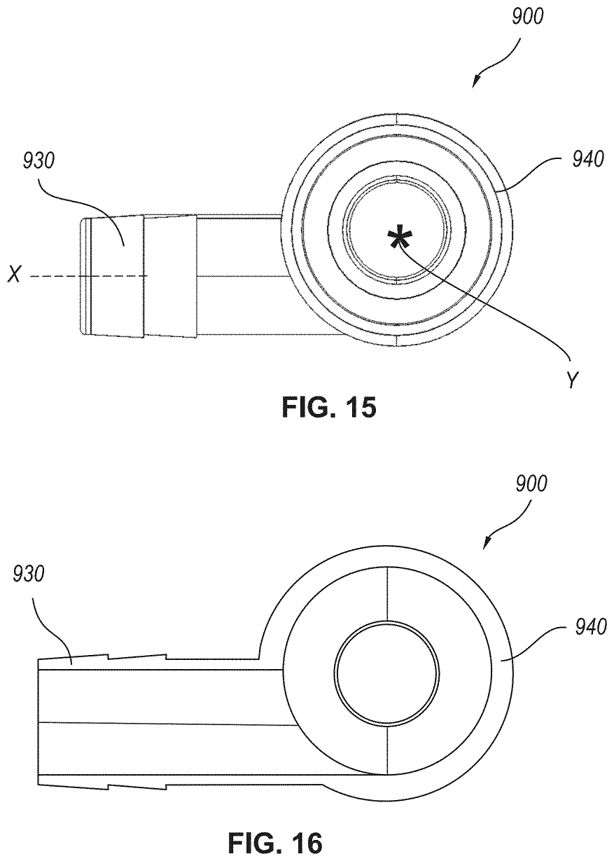

As shown in at least FIG. 14, in some embodiments, the first and second arms may extend substantially perpendicular to one another. As shown in FIG. 15, in such embodiments, a longitudinal axis X of the first arm may be laterally offset from a longitudinal axis Y of the second arm. As shown in FIGS. 15 and 16, in some embodiments, the first arm may be disposed tangentially to the second arm. In this regard, as shown in FIG. 16, the first lumen of the first arm 930 may connect with the second lumen of the second arm 940 tangentially. In some embodiments, this tangential relationship between the first and second arms may cause the blood flow entering the hub through the lumen to wash out the first lumen before it enters the shared lumen. In some embodiments, this may prevent blood from stagnating within the hub and may minimize thrombosis.

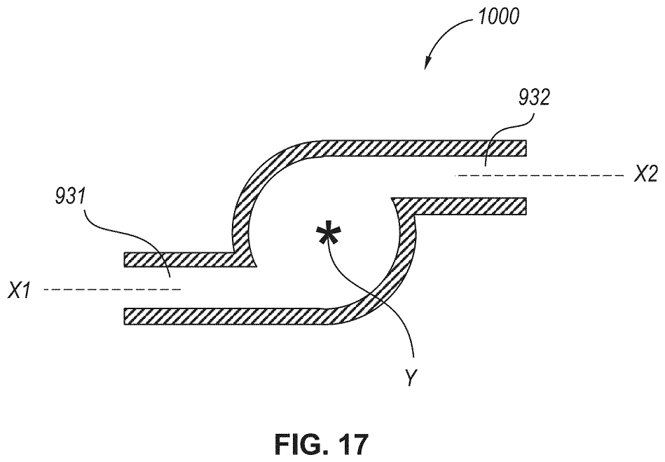

In some embodiments, as shown in FIG. 15, for example, the hub may include a single blood flow inlet for blood to enter the hub. As disclosed herein, this may create a circular flow to allow the blood to wash out the first lumen before entering the shared lumen. In other embodiments, as shown in FIG. 17, the hub may include first inlet 931 and second inlet 932 for blood flow into the hub. In such embodiments, each of the first and second inlets may include an arm, such as those disclosed herein, with a lumen.

As with FIG. 17, in some embodiments, each of the first inlet 931 and second inlet 932 may have a tangential relationship relative to the second arm (not shown). As shown in FIG. 17, in some embodiments, the first and second inputs may have first and second longitudinal axes X1, X2 that are each laterally offset from a longitudinal axis Y of the second arm. In some embodiments, as also shown in FIG. 17, the first and second longitudinal axes X1, X2 of the first and second inlets may be parallel and offset relative to one another.

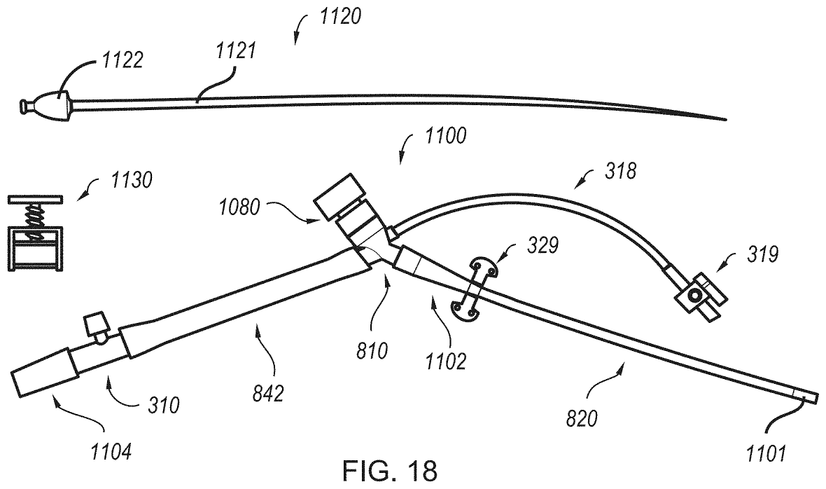

Referring to FIG. 18, an embodiment of another access system 1100 can be seen. As shown, hub 810 may be operably coupled to a cannula 820. The cannula may include a reinforcement cage 1101 at the distal end. The cannula also may include a coupling 1102 at the proximal end for connecting to the hub. A fixation feature 329 may be attached to the cannula such as to attach the access system to a patient. Tubing 318 (e.g., a high flow side port) may be used to couple the hub and a valve 319. Flexible tubing 842 (e.g., perfusion tubing) may be removable coupled to the hub (and specifically to the second lumen). A connector 841 (such as a 3/8″ barbed connector) may be coupled to a proximal end of tubing 1103. A tubing cap 1104 may be coupled to the connector. A clamp 1130 may be used, e.g., to control flow through tubing 1103.

A dilator 1120, including a tubular member 1121 attached coupled to a dilator handle 1122, may be present. In some embodiments, as disclosed herein, the tubular member may pass through an arm of the hub 200 into the cannula, while the dilator handle remains proximal to the hub 200. For example, in some embodiments, the tubular member of the dilator hub may remain be passed through a valve coupled to a proximal end of the hub, passing into the first arm and into the cannula. In some embodiments, the dilator may be used to facilitate insertion of a medical device into the patient (e.g., via the first arm).

In some embodiments, as shown in FIG. 18, the proximal end of the hub 810 may be coupled to one or more additional components. For example, the hub may include a Touhy Borst valve 1080 coupled to the proximal end of the first arm. In such embodiments, the Touhy Borst valve may serve as a redundant leak-protection feature.

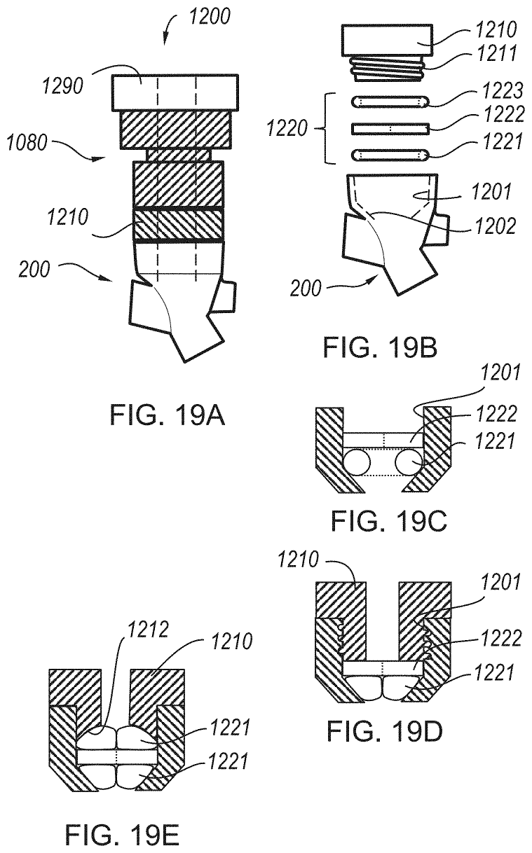

Referring to FIG. 19A, in some embodiments, a proximal end of the hub 200 may be coupled to a first cap 1210. In some embodiments, a Touhy Borst valve 1080 also may be coupled to the first cap 1210. In some embodiments, a dust cap 1290 may be coupled to the first cap or the Touhy Borst valve. For example, the dust cap may be removably attached to the Touhy Borst valve unless/until a medical device is insertable through the valve.

In some embodiments, a valve may be formed from two or more components sandwiched between the hub and the first cap or the Touhy Borst valve, or between the first cap and the Touhy Borst valve. In FIG. 19B, for example, the valve 1220 is shown as using three components—a first O-ring 1221, a silicone valve 1222 (note, this may be replaced by any other appropriate valve, and may include two or more layers, and may include, e.g., urethane foams, etc.), and an optional second O-ring 1223. In some embodiments, the hemostatic valve may be proximal to the first O-ring. In some embodiments, the hemostatic valve may be distal to the first O-ring. In some embodiments, the hemostatic valve may be positioned between a first and a second O-ring. In FIG. 19B, the first cap 1210 and hub 200 are referred to for forming the outer elements of the “sandwich”, but it will be understood that the hub and the Touhy Borst valve, or the first cap and the Touhy Borst valve could be utilized as well.

In some embodiments, the first cap may be removable coupled to the hub. For example, the first cap 1210 is shown as having a threaded portion 1211 that will interface with a surface (such as an inner surface 1201) of the hub. In some embodiments, the first cap may be permanently coupled to the hub. For example, the first cap may be adhered or welded to the hub.

Referring to FIG. 19C, in some embodiments, the silicone valve and the first O-ring may be placed into the hub, such that the first O-ring 1221 is prevented from moving distally by a portion 1202 of hub and prevented from moving proximally by the silicone valve 1222. Referring to FIG. 19D, when compressed (e.g., by the first cap being tightened down onto the hub), the silicone valve may press down on the O-ring, deforming it by pressing it against the portion 1202 of the hub and holding it in place. This deformation may allow the O-ring to act as a first barrier against the pressure of blood which may enter into the hub. As shown in FIG. 19E, this same concept may apply when two O-rings are present. The distal end 1212 of the first gap (shown here as having a concave shape) may press down on the second O-ring 1223, which deforms as it presses into the silicone valve 1222, which also compresses the first O-ring 1221 as described with respect to FIG. 19D. The deformation of the second O-ring may allow it to support the back of the silicone valve as it—and the first O-ring—act as barriers against the pressure of blood in the hub.

Those skilled in the art will recognize or be able to ascertain using no more than routine experimentation many equivalents to the specific embodiments of the invention described herein. Such equivalents are intended to be encompassed by the following claims.

Embodiments of the present disclosure are described in detail with reference to the figures wherein like reference numerals identify similar or identical elements. It is to be understood that the disclosed embodiments are merely examples of the disclosure, which may be embodied in various forms. Well known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

1. A hub for an access device, comprising:
a first arm with a first lumen extending from a proximal end to a distal end, the distal end being configured to be operably coupled to a cannula,
wherein a central axis of the first lumen at the distal end forms an angle with the central axis of the first lumen at the proximal end; and
wherein the first arm being configured to be operably coupled to a second arm, the second arm comprising a second lumen extending therethrough.

2. The hub according to claim 1, wherein the second arm is coupled to the hub.

3. The hub according to claim 2, wherein the second arm is removably coupled to the hub.

4. The hub according to claim 1, wherein:
a protrusion, a depression, or both is within the first lumen;
a protrusion, a depression, or both is with the second lumen; or a combination thereof.

5. The hub according to claim 1, further comprising a plug configured to be removably inserted into a proximal end of the first lumen.

6. The hub according to claim 1, further comprising one or more dilators that are configured to extend from the proximal end of the first lumen less than an entire length of the first lumen.

7. The hub according to claim 1, further comprising a plug configured to be removably inserted into a proximal end of the second lumen.

8. A hub for an access device, comprising:
a first arm with a first lumen extending from a proximal end to a distal end, the distal end being configured to be operably coupled to a cannula,
wherein the first arm being configured to be operably coupled to a second arm, the second arm comprising a second lumen extending therethrough; and
wherein:
a protrusion, a depression, or both is within the first lumen;
a protrusion, a depression, or both is with the second lumen;
or a combination thereof.

9-20. (canceled)

21. A hub for an access device, comprising:
a first arm with a first lumen extending from a proximal end to a distal end, the distal end being configured to be operably coupled to a cannula;
a second arm operably coupled to the first arm, the second arm comprising a second lumen extending therethrough; and
one or more dilators extending from the proximal end less than an entire length of the first lumen, wherein the one or more dilators is configured to fill at least a portion of space in the first lumen at or near the proximal end of the first lumen.

22-23. (canceled)

24. The hub according to claim 21, further comprising a plug configured to be inserted into a proximal end of the second lumen.

25. The hub according to claim 21, further comprising a plug configured to be inserted into the first lumen, wherein the plug includes a slit through which a medical device may be inserted into the first lumen.

26-27. (canceled)

28. The hub according to claim 21, wherein the hub further comprises a third lumen operably connected to the first lumen, the second lumen, or both.

29-35. (canceled)

36. The hub according to claim 21, wherein a longitudinal axis of the first arm is laterally offset from a longitudinal axis of the second arm.

37. The hub according to claim 1, wherein the central axis of the first lumen at the distal end forms a second arm angle with a central axis of the second lumen at the proximal end, where the angle is 15-30 degrees.

38. The hub according to claim 1, further comprising a cap coupled to the proximal end of the first arm.

39. The hub according to claim 38, further comprising an O-ring and a silicone valve positioned between a portion of the cap and a portion of the first arm.

40. The hub according to claim 39, further comprising a second O-ring disposed between the silicone valve and the cap.

41. The hub according to claim 38, wherein the cap is a Touhy Borst valve.

42. An access device comprising:
a cannula having a proximal end and a distal end, the cannula having a joint lumen therethrough; and
a hub according to claim 1, configured to be operably coupled to a proximal end of the cannula.

43. The access device according to claim 42, further comprising a second arm configured to be removably coupled to the hub, the second arm having a second lumen extending therethrough, the second lumen configured to be operably coupled to the first lumen through an opening in the hub.

44. The access device according to claim 42, further comprising a tubular extension capable of being removably coupled to the proximal end of the cannula and to the hub.

45. The access device according to claim 44, wherein the tubular extension is also configured to be capable of being removably coupled to a proximal end of a second arm.

46-62. (canceled)

63. A method for using an access device, comprising:
providing an access device according to claim 42;
inserting the cannula of the access device into a patient; and
inserting a medical device through a hub of the access device and into the patient, and/or oxygenating blood with an extracorporeal membrane oxygenation (ECMO) device operably coupled to the cannula through a second arm, an alternate connector subsystem, or both.

64-66. (canceled)

67. A kit comprising:
an access device according to claim 42;
an extracorporeal membrane oxygenation (ECMO) device configured to be operably coupled to a cannula of the access device; and
a medical device configured to be inserted through a hemostatic valve, a first lumen, and a joint lumen of the access device.

68. (canceled)
